(19) United States
(12) Patent Application Publication
(10) Pub. No.: US 2008/0145925 A1
(43) Pub. Date: Jun. 19, 2008

(54) Cell Culture Chamber

(76) Inventors: Yasuyuki Sakai, Tokyo (JP); Teruo Fujii, Tokyo (JP); Serge Ostrovidov, Tokyo (JP); Hiroaki Inui, Koriyama-shi (JP); Jinji Mizuno, Koriyama-shi (JP)

Correspondence Address: KENYON & KENYON LLP, ONE BROADWAY, NEW YORK, NY, 10004, US

(21) Appl. No.: 11/885,889

(22) PCT Filed: Dec. 8, 2005
(86) PCT No.: PCT/JP05/22524

§ 371 Date: Sep. 6, 2007

(30) Foreign Application Priority Data
Mar. 8, 2005 (JP) 2005-064074

(51) Int. Cl. C12M 1/12

(52) U.S. Cl. 435/297.2

(57) ABSTRACT

A cell culture chamber includes: a top compartment and a bottom compartment that are separated by a semi-permeable membrane inside a supporting body; a culture solution supply flow path that supplies culture solution to an interior of the bottom compartment; a culture solution discharge flow path that discharges culture solution from the interior of the bottom compartment; a circulation flow path that is used to circulate fluid within the interior of the top compartment; and a cell culturing portion is formed inside the top compartment by a sieve structure that is formed by aperture portions through which a fluid is able to pass but through which cultured cells cannot pass, and by wall portions.

### BACKGROUND OF THE INVENTION

1. Field of the Invention

The present invention relates to a cell culture chamber.

2. Description of Related Art

In-vitro fertilization exists as one of the techniques used in the fields of animal husbandry and reproduction treatment (especially in infertility treatment). Generally, in-vitro fertilization involves acquiring an ovum and performing in-vitro maturation if the ovum does not develop. After the in-vitro fertilization has been performed, the obtained fertilized ovum is cultured, and is then grown to a developmental stage which is suitable for transplanting. The ovum is then transplanted into a womb.

However, the conception success rate achieved using in-vitro fertilization is not necessarily high, and in the case, for example, of human in-vitro fertilization, the conception success rate still remains at between 25 to 35% since the world's first such childbirth 27 years ago in Britain. Because of this, for various reasons such as the fact that in-vitro fertilization is not covered by health insurance inside Japan, an improvement in the conception success rate is desired.

If the conception success rate is to be improved, then the aforementioned fertilized ovum culturing process is most important. Conventionally, the culturing of fertilized ova is achieved by performing in-vitro culturing in a static, closed environment using, for example, a method in which an approximately 500 μL culture solution is placed in a well on a culturing plate and then culturing a fertilized ovum in this culture solution, or a method in which approximately 20 μL micro droplets are placed in a well on a culturing plate and the surfaces of the micro droplets are then covered with mineral oil with the fertilized ovum then being placed inside this (see Non-Patent Document 1: “Method of Culturing Cells Having a Reproduction Function”, Sugawara, Ogawa (Ed.), Japan, Academic Publishing Center, June 1993, p. 25-153.).

However, when static closed-environment culturing is performed, the development efficiency is poor due to cell fragmentation and the like, and it has not been easy to obtain high-quality fertilized ova with any degree of frequency.

One of the reasons for it not being possible to obtain high-quality ova is considered to be the fact that the culturing environment is considerably different from the development environment inside a human body. Namely, generally, it is known that, after an ovum has been discharged from an ovary it transits through the ovarian duct as it matures. Initial development commences immediately after fertilization and during the 2 to 8 cell period the fertilized ovum passes through a morula stage so as to form a blastocyst, and then becomes implanted in the endometrium. The endometrium has a polarity in which the tissue has a layer structure formed by a layer of endometrium cells, a layer of interstitial cells, and the like, and the physicochemical environment and biological environment of the endometrium such as the fact that internal fluids are flowing through the womb and the interior of the oviduct are considerably different from those present in the above described conventional culturing methods.

Moreover, in order to obtain high quality fertilized ova, it is thought that it is extremely important to control the culturing environment such as by supplying nutrient components and oxygen, and removing waste matter and the like. However, in static closed-environment culturing, it is difficult to perform this type of control.

In contrast, one cell culturing method that is used conventionally is a method in which cells are adhered to the flat surface of a culturing vessel such as a Petrie dish, and by circulating a continuous flow of a culture solution containing nutrient components and oxygen to these cells, both the supplying of nutrient components and the removal of waste matter can be performed simultaneously. In this method, because the nutrient components and oxygen contained in the culture solution are dispersed through the cell layer and are supplied to the individual cells, while the waste matter conversely transits through the culture solution and is removed, the cells can be cultured continuously with their vitality maintained for an extended length of time.

However, in this type of method as well, it is difficult to satisfactorily reproduce the environment inside a human body, and it has not been easy to frequently obtain good quality fertilized ova. Moreover, when the cells being cultured are introduced into a vessel in order for the flow of culture solution to be circulated thereto, and also during culturing, and also when cultured cells are being recovered, there are cases when cells are lost, and great care is required in order to culture precious fertilized ova.

The present invention was conceived in view of the above described circumstances and it is an object thereof to provide a cell culture chamber that makes it possible to culture cells in an environment that closely resembles that found in a human body, and that enables operations such as introducing and recovering cells to be performed easily.

### SUMMARY OF THE INVENTION

In order to achieve the above described objectives, the present invention employs the following structure.

Namely, the cell culture chamber of the present invention includes: a top compartment and a bottom compartment that are separated by a semi-permeable membrane; a culture solution supply flow path that supplies culture solution to an interior of the bottom compartment; a culture solution discharge flow path that discharges culture solution from the interior of the bottom compartment; a circulation flow path that is used to circulate fluid within the interior of the top compartment; a supporting body that houses within it the top compartment, the bottom compartment, the culture solution supply flow path, the culture solution discharge flow path, and the circulation flow path; a cell culturing portion that is provided within the top compartment and whose periphery is surrounded by a sieve structure that has aperture portions through which the fluid is able to pass but cultured cells are not able to pass; and a guide that is connected to the cell culturing portion and is used to introduce and recover the cultured cells.

According to the cell culture chamber of the present invention, it is possible to culture cells in an environment that closely resembles that found in a human body, and that enables operations such as introducing and recovering cells to be performed easily.

Because the culturing of cells in an environment that closely resembles that found in a human body is possible, the quality of obtained cells is high and the development of fertilized ova, for example, can proceed in an excellent manner. As a result, the conception success rate from in-vitro fertilization can be improved.

### BRIEF DESCRIPTION OF THE DRAWINGS

FIG. 1 is a top view showing a first embodiment of the cell culture chamber of the present invention.

FIG. 2 is a vertical cross-sectional view at a position A-A′ in the cell culture chamber shown in FIG. 1.

FIG. 3 is a vertical cross-sectional view at a position B-B′ in the cell culture chamber shown in FIG. 1.

FIG. 4 is a view showing an example of a process to manufacture a cell culture chamber.

FIG. 5 is a top view showing a second embodiment of the cell culture chamber of the present invention.

FIG. 6 is a vertical cross-sectional view at a position C-C′ in the cell culture chamber shown in FIG. 5.

FIG. 7 is a vertical cross-sectional view at a position D-D′ in the cell culture chamber shown in FIG. 5.

FIG. 8A is a schematic structural view showing an example of a cell culturing apparatus that employs the cell culture chamber of the present invention.

FIG. 8B is a schematic structural view showing an example of a cell culturing apparatus that employs the cell culture chamber of the present invention.

FIG. 9 is a graph showing a temporal change in the incidence rate for blastocysts in Experimental example 1.

FIG. 10 is a graph showing total cell numbers and mean ICM cell numbers in Experimental example 1.

FIG. 11 is a graph showing a temporal change in the incidence rate for blastocysts in Experimental example 2.

FIG. 12 is a graph showing mean total cell numbers and mean ICM cell numbers in Experimental example 1.

### DETAILED DESCRIPTION OF THE INVENTION

Embodiments of the cell culture chamber of the present invention will now be described based on the drawings.

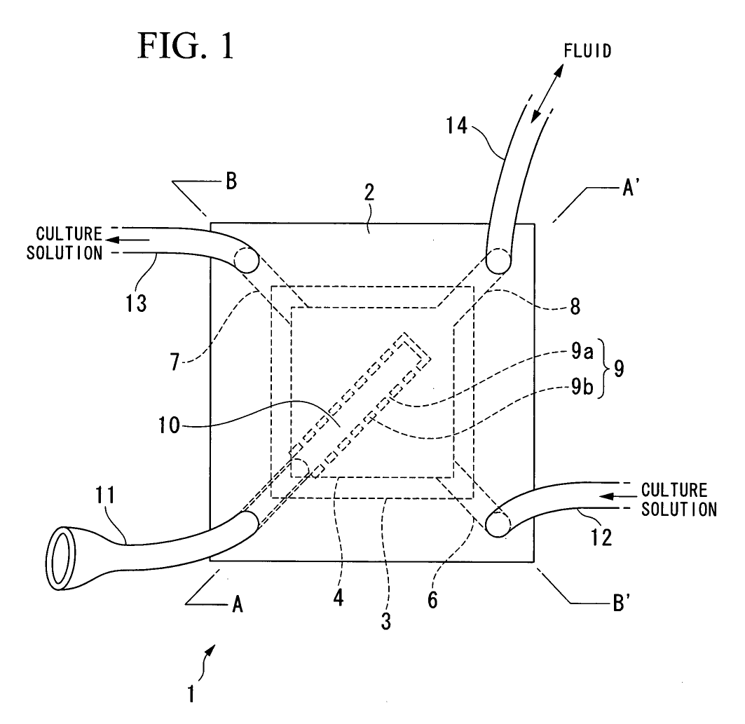

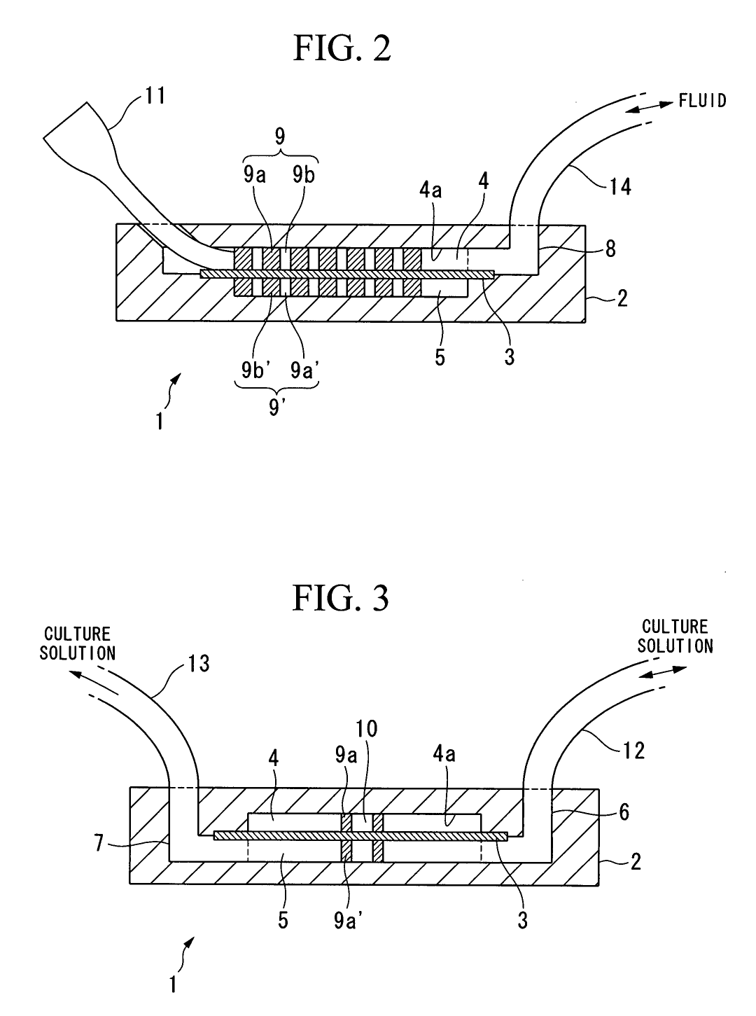

FIGS. 1 to 3 show a first embodiment of the cell culture chamber of the present invention. FIG. 1 is a top view showing a cell culture chamber 1 of the present embodiment, FIG. 2 is a vertical cross-sectional view at a position A-A′ in FIG. 1, and FIG. 3 is a vertical cross-sectional view at a position B-B′ in FIG. 1.

The cell culture chamber 1 of the present embodiment has a top compartment 4 and a bottom compartment 5 that are separated by a semi-permeable membrane 3 inside a supporting body 2, a culture solution supply flow path 6 that supplies culture solution to the interior of the bottom compartment 5, a culture solution discharge flow path 7 that discharges culture solution from the interior of the bottom compartment 5, and a circulating flow path 8 that is used to circulate fluid to the interior of the top compartment 4.

A cell culturing portion 10 is formed inside the top compartment 4 by sieve structures 9 that each have a U-shaped structure (i.e., a rectangular shaped having one end open) that is formed by aperture portions 9a through which a fluid is able to pass, but through which cultured cells, namely, those cells that are to be cultured in the cell culturing chamber cannot pass, and by wall portions 9b.

A guide 11 that is used to introduce and recover cultured cells is connected to the cell culturing portion 10.

Moreover, in the cell culturing chamber 1, tubes 12, 13, and 14 are connected respectively to the culture solution supply flow path 6, the culture solution discharge flow path 7, and the circulating flow path 8.

The material that constitutes the supporting body 2 is not particularly restricted provided that it is compatible with cultured cells.

Because oxygen from the outside air passes through the supporting body and is supplied to the cell culturing portion 10 and the culture solution inside the cell culture chamber 1, examples of such preferred materials include oxygen permeable materials that allow oxygen to permeate. An optional known oxygen permeable material can be used for the oxygen permeable materials provided that it is compatible with the cultured cells, and examples thereof include biocompatible oxygen permeable materials that are used in oxygen permeable contact lenses and the like. Materials that have transparency are particularly favorable as these allow the cell culturing inside the cell culturing portion 10 to be observed from the outside.

A specific example of an oxygen permeable material is biocompatible silicone rubber. Polydimethylsiloxane (referred to below as PDMS) is particularly preferable as, in addition to it being biologically compatible, it is transparent and oxygen permeable and is also an inexpensive material.

A material having a hole diameter which the cells are unable to infiltrate but that allows other substances (for example, components (i.e., nutrient components and oxygen) in the culture solution circulated to the bottom compartment 5, and waste matter and the like from the cultured cells inside the top compartment 4) to be replaced is used for the semi-permeable membrane 3. An upper limit for the hole diameter of the semi-permeable membrane 3 is preferably less than 3 μm, and is more preferably not more than 1 μm. A lower limit is preferably not less than 0.4 μm as this allows a fast replacement rate for the other substances.

The material that is used for the semi-permeable membrane 3 is not particularly restricted provided that it is compatible with the cultured cells. For example, generally, commercially available materials that are used for dialysis membranes, precise filtration, and the like can be used for the semi-permeable membrane. Specific examples thereof include polyethylene, polycarbonate, polyester, polytetrafluoroethylene (referred to below as PTFE) and the like.

The thickness of the semi-permeable membrane is preferably within a range of 10 to 20 μm in order to increase the substance replacement rate to the maximum.

Culture solution is circulated during cell culturing via the culture solution supply flow path 6 and the culture solution discharge flow path 7 to the bottom compartment 5. Namely, culture solution is supplied via the culture solution supply flow path 6 to the interior of the bottom compartment 5, and supplied culture solution is circulated through the interior of the bottom compartment 5 and is discharged from the bottom compartment 5 via the culture solution discharge flow path 7. At this time, nutrient components and oxygen within the culture solution pass through the semi-permeable membrane 3 and transit to the top compartment 4. As a result, nutrient components and oxygen are supplied from a fixed direction to the cultured cells within the cell culturing portion 10, and it is possible to create an environment that is similar to the actual environment inside a human body in which nutrient components and oxygen and the like are supplied from blood vessels and the like.

During cell culturing, fluid inside the top compartment 4 is either constantly or intermittently supplied to or circulated within the top compartment 4 via the circulating flow path 8. By supplying the fluid within the top compartment 4 or by circulating this fluid, a flow is added to the cultured cells within the cell culturing portion 10, which results in the environment within the cell culturing portion 10 approximating the actual environment within a human body (for example, a flow of internal fluids is present at the womb internal surface and at the oviduct internal surface), and high-quality cultured cells can be cultured. Moreover, it is also possible to monitor the culturing environment (i.e., the pH, the glucose concentration, the concentration of physiologically active substances and the like) of cultured cells of fertilized ova and the like via the circulating flow path 8.

The fluid that is circulated in the top compartment 4 should not have any adverse effects on the cultured cells, and a culture solution is particularly preferable for this.

As has been described above, by causing a fluid to flow inside the top compartment 4 via the circulating flow path 8 while also causing culture solution to circulate inside the bottom compartment 5 via the culture solution supply flow path 6 and the culture solution discharge flow path 7 of the cell culturing chamber 1, it is possible to culture cells in an environment that resembles the environment inside a human body even more closely.

The thickness of the top compartment 4 (i.e., the distance from the semi-permeable membrane 3 to an upper inner surface 4a of the top compartment 4) should have a bottom limit that is larger than the size of the cultured cells, and it is particularly preferable for it to be 1.5 times the size of the cultured cells.

The upper limit of the thickness of the top compartment 4 is not particularly restricted, however, it is preferably within 1 mm. If it is within 1 mm, then the quality of the cultured cells is particularly high, and when, for example, fertilized ova are being cultured, there is a high probability of normal development proceeding, and the conception success rates can be further improved. If the thickness of the top compartment 4 is within 1 mm, then this enables the concentration of nutrient components and oxygen that are supplied from the culture solution circulating through the interior of the top compartment 5, and also the concentration of the various promoters that are supplied from feeder cells when co-culturing is performed (described below) to be kept at a high level, which is thought to closely resemble the actual environment within the human body. The thickness of the top compartment 4 is more preferably within 5 times the size of the cultured cells and even more preferably within 2 times the size of the cultured cells.

A cell culturing portion 10 is formed inside the top compartment 4 by sieve structures 9 that each have a U-shaped structure (i.e., a rectangular shaped having one end open) that is formed by aperture portions 9a through which a fluid is able to pass, but through which cultured cells cannot pass, and by wall portions 9b.

The size of the aperture portions 9a should be a size that allows a fluid to pass through but does not allow cultured cells to pass through, and is appropriately set in accordance with the size of the cells being cultured.

In particular, as is described below, when performing co-culturing with feeder cells, it is preferable for the size of the aperture portions 9 to be a size that does not allow the cultured cells to pass through but does allow feeder cells to pass through. As a result of this, as is described below, the dissemination and culturing of the feeder cells over the semi-permeable membrane 3 of the cell culturing portion 10 can be easily performed.

The wall portions 9b are shown in the drawings to be in contact with the semi-permeable membrane 3, however, the present invention is not limited to this and it is also possible for a gap to be formed between the wall portions 9b and the semi-permeable membrane 3 that allows a fluid to pass through but does not allow cultured cells to pass through.

In the cell culture chamber 1, a sieve structure 9′ that is formed by aperture portions 9a′ and wall portions 9b′ in the same way as in the top compartment 4 is also provided in the bottom compartment 5. In the bottom compartment 5, the sieve structure 9′ is not absolutely essential, however, as a result of the sieve structure 9 being present, the semi-permeable membrane can be stably supported.

In the cell culture chamber 1, it is preferable for feeder cells that are used for co-culturing together with the cultured cells to be adhered to the top compartment 4 side of the semi-permeable membrane 3. As a result of this, the environment within the top compartment 4 can be approximated even more closely to the actual environment within the human body, and excellent culturing can be achieved. Namely, the effects of various growth promoters that are released from the oviduct and endometrial tissue are received by a fertilized ovum, for example, after fertilization right up to implantation in order for development to progress. Because of this, by performing the culturing of fertilized ova with feeder cells from the endometrial tissue or the like adhering to the top of the semi-permeable membrane 3, it is possible to improve the proportion of fertilized ova that develop normally (i.e., the development ratio).

Here, in the present invention, the tem ‘co-culturing’ refers to a method in which cultured cells and somatic cells of the same type or a different type of animal are cultured simultaneously.

The type of feeder cell is decided in accordance with the type of cultured cell. For example, if the cultured cell is a fertilized ovum of a mammal, then it is preferable for the feeder cells to be somatic cells or tissue of the same type of mammal. Specific examples thereof include fibroblasts, reproductive organ original cells (endometrial cells, uterine tubal epithelium cells and the like), or tissue that is formed by these cells. In particular, when the cultured cells are those of a fertilized ovum, it is preferable for the feeder cells to be uterine tubal epithelium cells. Moreover, when the cultured cells are ES cells (described below), it is preferable for the feeder cells to be fibroblasts from the same type of mammal that have been rendered inactive.

The feeder cells may be adhered onto the semi-permeable membrane in the following manner. For example, prior to the culturing of the cultured cells, the bottom compartment 5 is firstly filled with culture solution which is left uncirculated, and culture solution (i.e., a feeder cell suspension) that contains the feeder cells is introduced into the top compartment 4 using the circulating flow path 8 and the guide 11, and without making any further modifications, culturing is performed in this closed state. As a result, the feeder cells are adhered onto the semi-permeable membrane 3.

Examples of the cultured cells that are cultured in the cell culturing portion 10 include cells originating from mammals such as humans, mice, cattle, and pigs.

Ova and fertilized ova are particularly favorable because they are valuable in various fields such as the fields of animal husbandry and reproductive medicine, and fertilized ova are particularly favorable. This is because the cell culture chamber of the present invention is able to culture cells in an environment that resembles the environment within the human body, and makes it possible to perform high quality ova maturation and high quality extra-somatic development of fertilized ova obtained through in-vitro fertilization.

The size of the ova and fertilized ova is normally approximately 130 μm in the case of humans, approximately 80 μm in the case of mice, and approximately 120 to 130 μm in the case of cattle and pigs.

The cell culture chamber of the present invention is particularly suitable for developing fertilized ova. Note that, after fertilization, the fertilized ova passes through the morula stage in which the cell number increases through ova segmentation through a two-cell stage, a four-cell stage, and an eight-cell stage, and develops into a blastocyst. The blastocyst is formed by a trophectoderm and an embryoblast inside the trophectoderm, and, in in-vitro fertilization, transplanting into the uterus is normally performed between the 4 to 8 cell stage and the blastocyst stage.

In the cell culture chamber of the present invention it is also possible to culture embryonic stem cells (referred to below as ES cells). ES cells are undifferentiated cells that are obtained from the aforementioned embryoblasts, and are grown in an undifferentiated state when culturing is performed using fibroblasts as the feeder cells and leukemia inhibiting factors (LIF) are added. Because all cells have the property of becoming differentiated, by changing the culturing conditions, it is anticipated that ES cell culturing will be able to be used in regenerative medicine in which ES cells are used to regenerate the desired tissue or cells which are then transplanted to a patient.

In the cell culture chamber of the present invention, because the volume of the top compartments 4 is extremely small, and the feeder cells and ES cells interact intimately with each other both directly and indirectly, it is conjectured that culturing will be possible in an undifferentiated state over an extended period of time.

The size of mammalian ES cells is normally approximately 10 μm at the longer axis and approximately 5 μm during bonding, and they are spherical bodies of approximately 5 to 10 μm when they have been peeled away using trypsin or the like and placed in a floating state.

A guide 11 that is used for the introduction and recovery of cultured cells is connected to the cell culturing portion 10.

In the present embodiment, the guide 11 is a tube that is attached to a side face of the aperture portion of the U-shaped (i.e., a rectangular shaped having one end open) sieve structure 9.

Prior to the start of culturing, a tube (not shown) is inserted into the guide 11, and cultured cells are introduced via this tube into the cell culturing portion 10.

During culturing, when a circulating flow is not applied in the top compartment 4, a cap is attached so as to seal an end portion 11a of the guide 11. At this time, it is preferable for a flow motion to be added to the fluid within the top compartment 4 via the circulating flow path 8. If the guide 11 is only used to introduce and recover cultured cells in this manner, it is difficult for cultured cells to be lost. Because of this, the guide 11 is favorable for the development of fertilized ova and the maturation of ova.

When a circulating flow is applied in the top compartment 4, the circulating flow can be achieved, for example, by using the tube 14 and the guide 11 respectively as the fluid supply flow path and the fluid discharge flow path. Using the guide 11 as a circulating flow path in this manner is favorable when a circulating flow is critical such as for ES cells and the like.

After culturing has ended, a tube (not shown) is once again inserted into the guide 11, and cultured cells can be recovered by recovering the fluid within the cell culturing portion 10 via this tube.

The inner diameter of the guide 11 should be large enough to allow the tube that is inserted into the guide 11 during introduction or recovery of cells to be inserted (i.e., the inner diameter is larger than the cultured cells), and the outer diameter should be not more than the thickness of the top compartment.

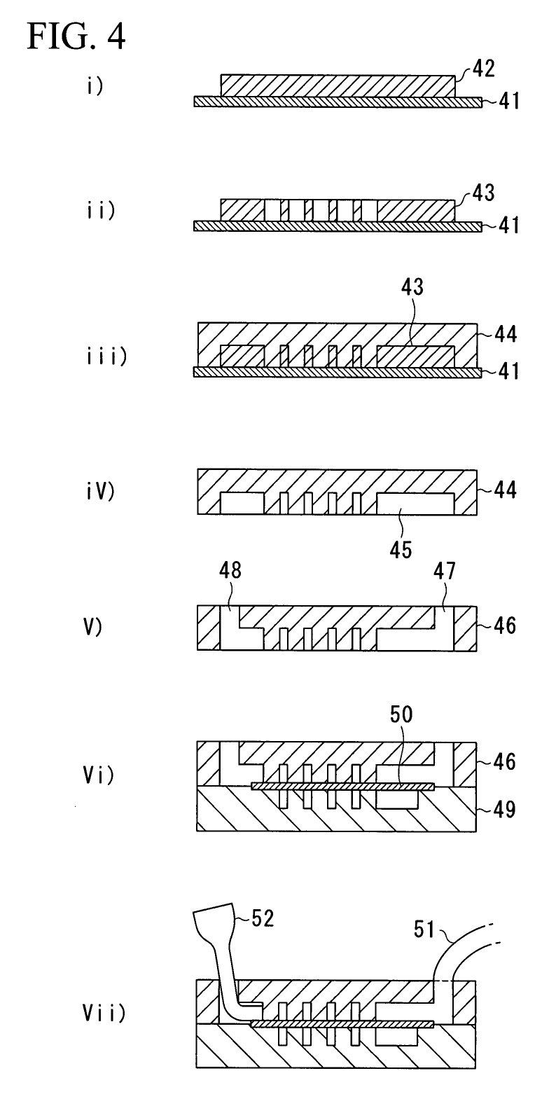

The cell culture chamber 1 can be manufactured, for example, in the manner shown in FIG. 4.

i) Firstly, a photoresist layer (for example, ‘SU-8’ (trade name) manufactured by MicroChem Corporation) 42 is formed by spin coating on a silicon substrate 41.

ii) The photoresist layer is then developed by exposing it via a predetermined mask pattern, so that a mold 43 for the top compartment is formed on the second substrate 41.

iii) An unpolymerized UV curing type or thermosetting type of uncured polymer is coated onto the obtained mold 43 so as to form a polymer layer 44, and this is then cured by UV irradiation or by applying heat.

iv) The cured polymer layer 44 is then peeled away, so that a polymer layer 44 is obtained on one surface of which is formed a recessed portion 45 having a sieve structure.

v) A hole (not shown) for the culture solution supply flow path, a hole (not shown) for the culture solution discharge flow path, a hole 47 for the flow movement applying flow path, and a hole 48 that is used to attach a guide are formed in the polymer layer 44 resulting in a top supporting body 46 being obtained.

vi) Separately, a bottom supporting body 49 is prepared in the same way as is described in i) to iv) above, and the top supporting body 46 and bottom supporting body 49 are adhered together via a semi-permeable membrane 50 such that recessed portions thereof are on the internal side.

vii) A tube 51 and a guide 52 are mounted in the holes formed in v) above thereby completing the cell culture chamber.

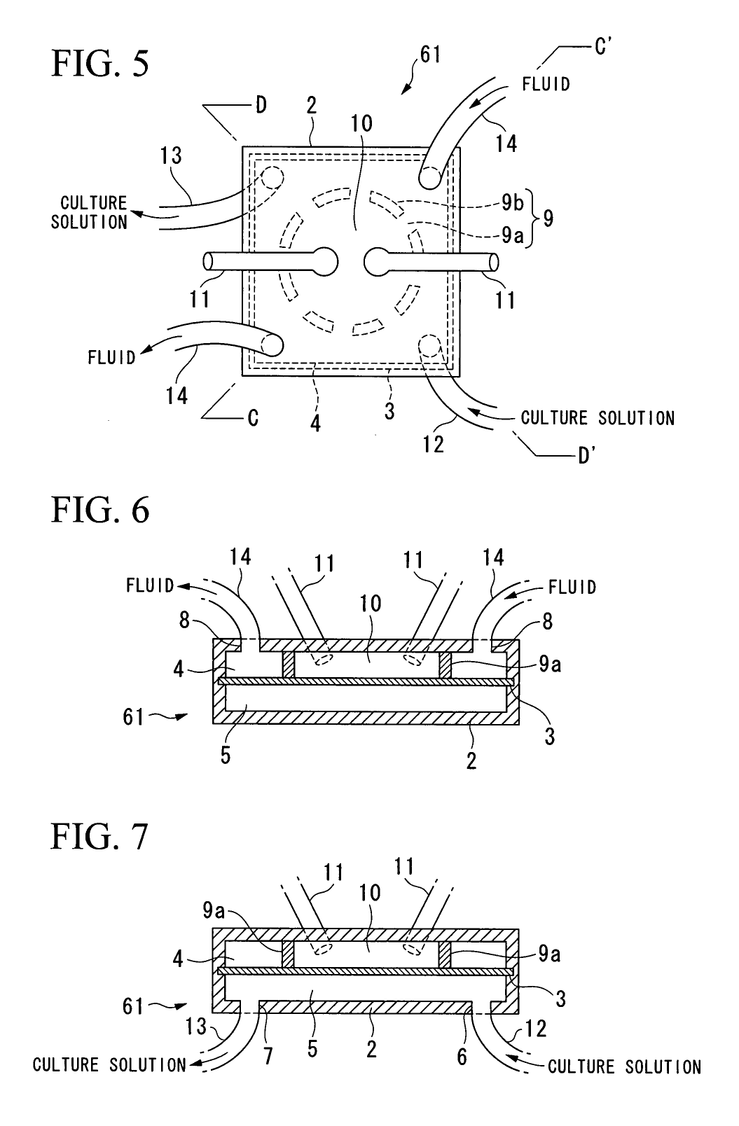

A second embodiment of the cell culture chamber of the present invention is shown in FIGS. 5 to 7. FIG. 5 is a top view of a cell culture chamber 61 of the present embodiment. FIG. 6 is a vertical cross-sectional view at a position C-C′ in FIG. 5. FIG. 7 is a vertical cross-sectional view at a position D-D′ in FIG. 5. Note that in the embodiment described below, component elements that correspond to those in the above described first embodiment are given the same symbols and a detailed description thereof is omitted.

The cell culture chamber 61 of the present embodiment differs from the first embodiment in that the shape of the sieve structure 9 is a circular cylinder shape, in that the sieve structure 9′ is not provided in the bottom compartment 5, in that the culture solution supply flow path 6 and the culture solution discharge flow path 7 are provided below the bottom compartment 5, in that the circulating flow path 8 is provided in two locations above the top compartment 4, and in that two guides 11 and 11 are connected from the top of the cell culturing portion 10.

The cell culture chamber of the present invention can be used as a cell culturing apparatus by causing a culture solution to circulate via a culture solution supply aperture and a culture solution discharge aperture.

The rate of the circulation of the culture solution is not particularly limited, and may be set appropriately in accordance with the cells being cultured.

Moreover, it is preferable for the culture solution being circulated to be replaced at least every three to four days in order to remove waste matter discharged in the culture solution and cell secreted material.

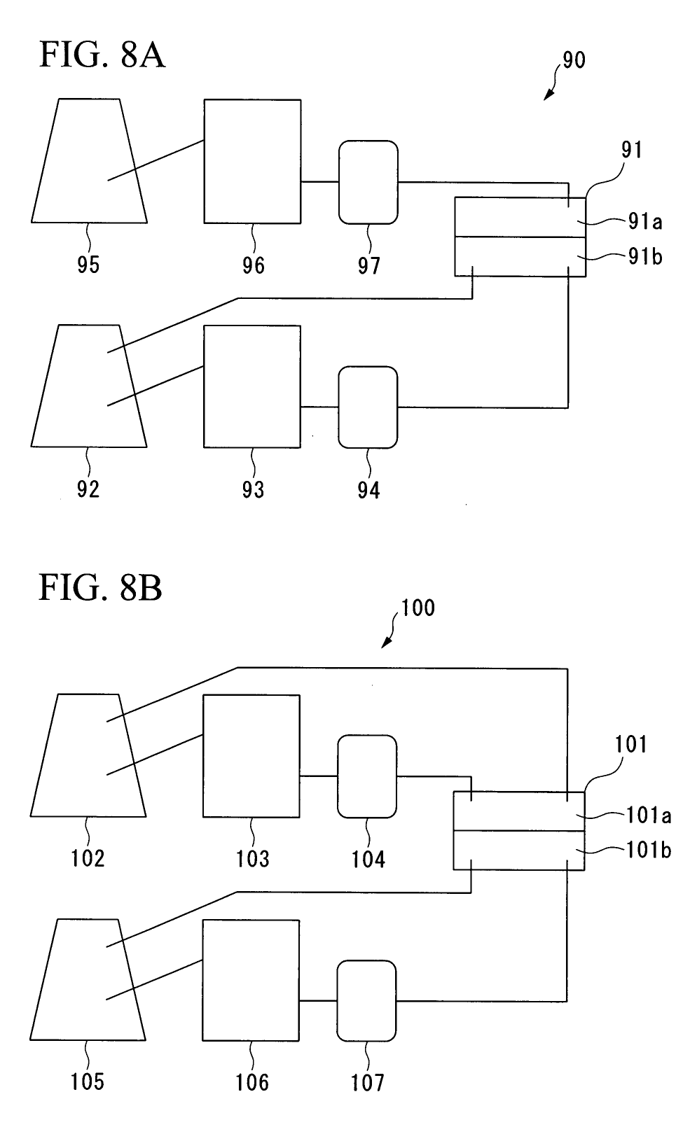

FIG. 8A and FIG. 8B are schematic structural views each showing an example of a cell culturing apparatus that uses the cell culture chamber of the present invention.

A cell culturing apparatus 90 shown in FIG. 8A is an example of a case in which a circulating flow is not provided in a top compartment 91a of a culture chamber 91, and a closed system circulating flow circuit is connected to a bottom compartment 91b. A culture solution tank 92, a peristaltic pump 93, and a bubble trap 94 are placed on the closed system circulating flow circuit that is connected to the bottom compartment 91b. A circuit provided with a fluid tank 95, a pump 96 that causes the fluid to flow, and a bubble trap 97 are provided in the top compartment 91a.

A cell culturing apparatus 100 shown in FIG. 8B is an example of a case in which a circulating flow is provided in a top compartment 101a of a cell culture chamber 101, and a closed system circulating flow circuit is connected to both a top compartment 101a and a bottom compartment 101b of the cell culture chamber 101. A fluid tank 102, a peristaltic pump 103, and a bubble trap 104 are placed on the closed system circulating flow circuit that is connected to the top compartment 101a. A culture solution tank 105, a peristaltic pump 106, and a bubble trap 107 are placed on the closed system circulating flow circuit that is connected to the bottom compartment 101b.

### EXAMPLES

### Manufacturing Example 1

### Manufacturing of a Cell Culture Chamber

A cell culture chamber having the configuration shown in FIG. 1 to FIG. 3 was manufactured following the process shown in FIG. 4.

Firstly, a silicon substrate was prepared and photoresist (trade name ‘SU-8 80;’ manufactured by MicroChem Corporation) was coated by spin coating onto this substrate. The substrate was then baked so as to form a photoresist layer. Next, the photoresist layer was exposed via a mask pattern and developed, and the pattern was then transferred onto a wafer thereby creating a mold. Uncured PDMS was then coated onto this mold so as to form a polymer layer, and this was then cured by UV irradiation. After the curing, the polymer layer was peeled off, thereby forming a top supporting body and a bottom supporting body that each had a recessed portion (10 mm×10 mm×200 μm) provided with a sieve structure on one surface thereof. Next, a hole for the culture solution supply flow path, a hole for the culture solution discharge flow path, a hole for the flow movement applying flow path, and a hole that is used to attach a guide were formed in the top supporting body, and the top supporting body and bottom supporting body were adhered together via a semi-permeable membrane (i.e., a polyester membrane having a hole diameter of 0.4 μm) such that recessed portions thereof were on the internal side. Thereafter, a tube and a guide were mounted in the respective holes, thereby creating a cell culture chamber (10 mm×10 mm×a thickness of 400 μm; a top compartment thickness of 200 μm and a bottom compartment thickness of 200 μm).

### Test Example 1

### Verification of Effects in a Co-Culturing System

Using the cell culture chamber manufactured in Manufacturing example 1 (i.e., made from PDMS; 10 mm×10 mm×a thickness of 400 μm; top compartment thickness of 200 μm) as Example 1, a mouse two-cell stage fertilized mice ova were co-cultured together with feeder cells, and the improvement effect in the development efficiency was confirmed. Mouse endometrial cells (MEC) were used for the feeder cells.

‘Static culturing on a plate’ was performed for Comparative example 1. Namely, feeder cells were sown over a culture dish and culturing was performed in conjunction with these feeder cells.

‘Static culturing on a membrane’ was performed for Comparative example 2. Namely, using a cell culture insert (CCI) having a membrane structure in the bottom portion of its body, fertilized ova were co-cultured together with the MEC on this bottom portion membrane.

Note that 50 μM of β mercaptoethanol was added in advance to the culture solution in order to prevent developmental damage caused by oxidation due to oxygen present in the vapor phase.

Culturing was performed following the process described below using the cell culturing apparatus shown in FIG. 8A.

All of the components constituting the cell culturing apparatus 100 were sterilized in advance using an autoclave.

Prior to the introduction of the feeder cells (i.e., endometrial cells) and the fertilized ovum, the cell culture chamber 101 was washed with Dulbecco's phosphate buffer solution. Next, after 0.03% I-type collagen aqueous solution (manufactured by Nitta Gelatins) was introduced, the cell culture chamber was left static for one hour in an incubator having a carbon dioxide concentration of 5% and an oxygen density of 19.5% at between 34 and 36° C., so that a semi-permeable membrane surface was coated thereon.

When the bottom compartment 5 was filled with culture solution, the endometrial cells were introduced by syringe reciprocation into the top compartment 4 from the circulation flow path 8 and the guide 11. In this state, they were left static overnight inside the incubator. As a result, it was recognized that cells were adhering to the surface of the semi-permeable membrane of the cell culture chamber.

Next, culture solution was circulated at a circulation flow rate of 150 μl/min inside the closed system circulation circuit that is connected to the bottom compartment 101b, and culturing was performed in the culturing conditions described below.

### [Culturing Conditions]

The culture solution was obtained by adding 5% by mass of human-derived blood serum as well as 50 μM of β mercaptoethanol, penicillin, streptomycin, and gentamycin to MEM-α (manufactured by GIBCO). Note that at this time, it is also possible to use a culture solution for another fertilized ovum in accordance with the desired objective as the culture solution.

Culturing Method

Culturing was performed in a CO₂ incubator (5% CO₂ and 95% air, 37° C., humidity saturation).

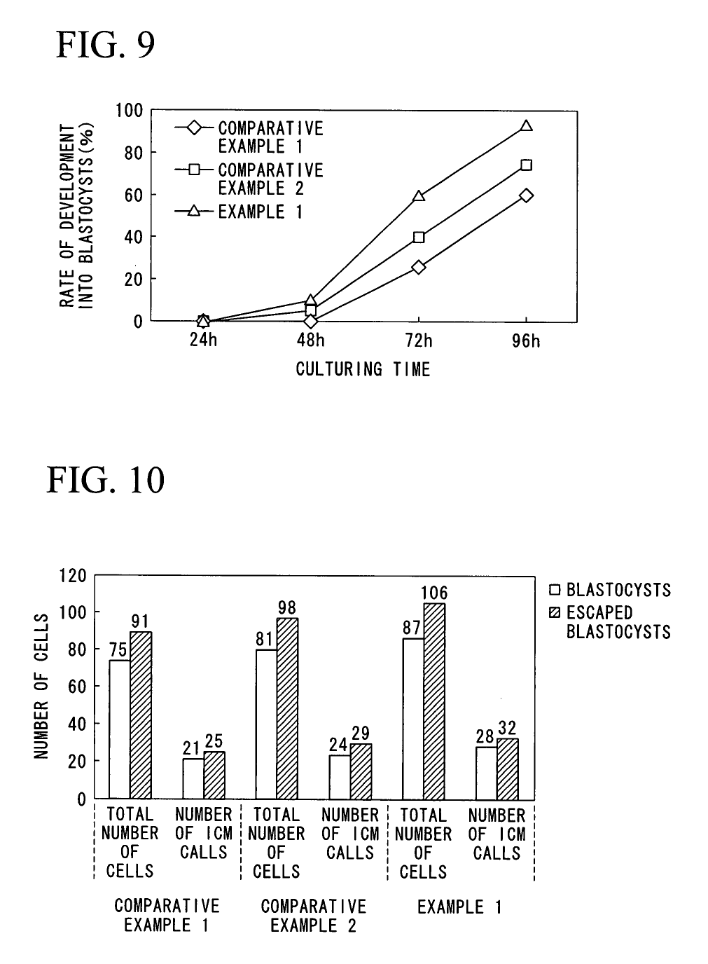

From 24 hours after the commencement of culturing, monitoring and recording of the development of the fertilized ova into a blastocyst were performed every 24 hours until 96 hours had elapsed. A ratio of the number of fertilized ova that had developed into blastocysts relative to the number of fertilized ova at the start of culturing (i.e., fertilized ova at the mouse two-cell stage) was determined as a development ratio. The results thereof are shown in FIG. 9.

In addition, the total number of cells constituting the obtained mouse blastocysts and the number of embryoblast cells inside the blastocysts (i.e., the number of ICM cells) were measured using dual fluorescent staining. Moreover, the same measurements were made for escaped blastocysts (i.e., blastocysts that have escaped from the pellucid zone (a capsule covering the fertilized ovum)). The results thereof are shown in FIG. 10.

From FIG. 9 it is clear that the period until the blastocyst stage was reached in Example 1 was significantly shorter than in Comparative examples 1 and 2.

From FIG. 10 it is clear from the measurement results of the number of cells at the points when the blastocyst stage and escaped blastocyst stage were reached in the respective systems that the total number of cells and the number of ICM cells were significantly more in Example 1 than in Comparative examples 1 and 2. As a result, if the obtained blastocysts are transplanted into the uterus of a recipient mouse, an improvement in both the recipient female rate of conception and the offspring production rate can be expected.

In this manner, in a co-culturing system, using the cell culture chamber of the present invention, mice fertilized ova that have been cultured in a circulating flow in a co-culturing system have a higher rate of development into blastocysts, a greater number of cells, and a higher quality compared to the comparative examples. These results show that, using the cell culture chamber of the present invention, by performing circulatory culturing in a co-culturing system, the developmental capabilities of mammalian fertilized ova is increased markedly. This phenomenon may be said to coincide with our objective of externally recreating the environment within a human body.

### Test Example 2

### Verification of Effects without Co-Culturing

Other than the fact that co-culturing was not performed, in the same manner as in Test example 1, culturing using the cell culture chamber manufactured in Manufacturing example 1 (Example 2), ‘Static culturing on a plate’ (Comparative example 3), and ‘Static culturing on a membrane’ (Comparative example 4) were performed. The same evaluations were then made.

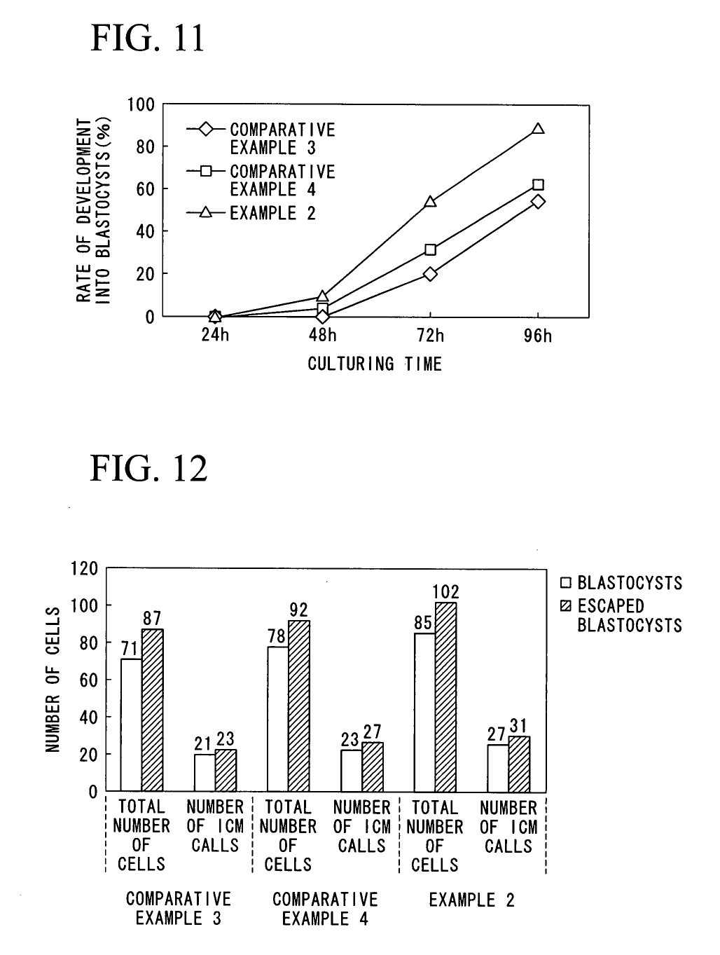

The rates of development into blastocysts are shown in FIG. 11. The total number of cells constituting the obtained blastocysts and the escaped blastocysts as well as the number of ICM cells are shown in FIG. 12.

From FIG. 11 it is clear that the period until the blastocyst stage was reached in Example 2 was significantly shorter than in Comparative examples 3 and 4.

From FIG. 12 it is clear that the total number of cells and the number of ICM cells were both significantly more in Example 2 than in Comparative examples 3 and 4. As a result, an improvement in both the corresponding recipient female rate of conception and the offspring production rate can be easily predicted.

In this manner, although the effects were inferior compared with the co-culturing system used in Test example 1, even in a non co-culturing system, fertilized mice ova that were cultured in a circulating flow using the cell culture chamber of the present invention had a higher rate of development into blastocysts, a greater number of cells, and a higher quality compared to the comparative examples. These results show that, using the cell culture chamber of the present invention, by performing circulatory culturing even in a non co-culturing system, the developmental capabilities of mammalian fertilized ova is increased markedly. This phenomenon may be said to coincide with our objective of externally recreating the environment within a human body.

What is claimed is:

1. A cell culture chamber comprising:
a top compartment and a bottom compartment that are separated by a semi-permeable membrane;
a culture solution supply flow path that supplies culture solution to an interior of the bottom compartment;
a culture solution discharge flow path that discharges culture solution from the interior of the bottom compartment;
a circulation flow path that is used to circulate fluid within the interior of the top compartment;
a supporting body that houses within it the top compartment, the bottom compartment, the culture solution supply flow path, the culture solution discharge flow path, and the circulation flow path;
a cell culturing portion that is provided within the top compartment and whose periphery is surrounded by a sieve structure that has aperture portions through which the fluid is able to pass but cultured cells are not able to pass; and
a guide that is connected to the cell culturing portion and is used to introduce and recover the cultured cells.

2. The cell culture chamber according to claim 1, wherein a thickness of the top compartment on the semi-permeable membrane is not more than 1 mm.

3. The cell culture chamber according to claim 1, wherein feeder cells that are used for co-culturing with the cultured cells are adhered onto the top compartment side of the semi-permeable membrane.

4. The cell culture chamber according to claim 3, wherein the cultured cells are fertilized ova, and the feeder cells are cells derived from reproductive organs or fibroblasts.

5. The cell culture chamber according to claim 3, wherein the cultured cells are embryonic stem cells, and the feeder cells are inactivated fibroblasts.

6. The cell culture chamber according to claim 1, wherein the supporting body is composed of polydimethylsiloxane.
